(19) United States
(12) Patent Application Publication
(10) Pub. No.: US 2022/0154153 A1
(43) Pub. Date: May 19, 2022

(54) NEW INHIBITORS OF LRRK2/PP1 INTERACTION

(71) Applicants: Université de Paris, Paris (FR); Sorbonne Universite, Paris (FR); Centre National de la Recherche Scientifique, Paris (FR); Institut National de la Sante et de la Recherche Medicale, Paris (FR)

(72) Inventors: Angelita REBOLLO, Paris (FR); Pierre TUFFERY, Moisselles (FR); Chang-Zhi DONG, L'Haÿ-les-Roses (FR)

(21) Appl. No.: 17/439,641

(22) PCT Filed: Mar. 21, 2020
(86) PCT No.: PCT/EP2020/057898

§ 371 Date: Sep. 15, 2021

(30) Foreign Application Priority Data
Mar. 22, 2019 (EP) 19305365.9

(51) Int. Cl. C12N 9/12; C07K 16/40

(52) U.S. Cl. CPC C12N 9/12; C12Y 207/11001; A61K 38/00; C07K 2319/00; C07K 16/40

(57) ABSTRACT

The present invention provides new inhibitors of LRRK2/PP1 interaction. The present invention relates to these inhibitors for use as medicament and more particularly to methods and pharmaceutical compositions for the treatment of neurodegenerative disorders, more particularly α-synucleinopathies.

### FIELD OF THE INVENTION

The present invention provides new inhibitors of LRRK2/PP1 interaction. The present invention relates to these inhibitors for use as medicament and more particularly to methods and pharmaceutical compositions related to the treatment of neurodegenerative disorders, more particularly α-synucleinopathies.

### BACKGROUND OF THE INVENTION

A common feature of neurodegenerative disorder is the loss of neuronal processes as neurite outgrowth and neuronal cell viability. Mutations in LRRK2 have been shown to induce reduction in neurite length and branching, tau aggregates formation and ultimately to lead to neuronal cell apoptosis (Mac Leod et al. 2006). Some have found that LRRK2 appears to be present in neuronal and glial inclusions in several neurodegenerative disorders, leading to the hypothesis that a common link may exist in the pathogenesis of these disorders (Miklossy et al., 2006).

More specifically, mutations in the gene encoding LRRK2 are known to be responsible for the genetic forms of Parkinson's disease (PD). However, these mutations are not systematically associated with the development of the disease, factors preventing the development of the disease may be present in subjects carrying these mutations. Nevertheless, a significant proportion of apparently isolated cases of Parkinson's disease originates from a dominant mutation of the LRRK2 gene, resulting in the substitution G2019S: in North Africa it is present in 37% of familial PD cases. This mutation was also found in 41% of apparently isolated cases of PD in subjects of North African origin. Such large proportions of mutants in cases of parkinsonism are also observed in other specific populations. Highlighting even more the importance of LRRK2 in PD, the literature reports that the clinical symptoms associated with LRRK2 mutations associated with Parkinson's disease can not be distinguished from those of sporadic cases.

The G2019S mutation, like other mutations responsible for the autosomal transmission of the disease, is linked to a hyperactivation of the kinase activity (autophosphorylation) of LRRK2 and this is why there are some inhibitors of LRRK2 kinase activity which are currently in clinical testing. However, literature mentions side effects for inhibitors formerly tested.

Some PD-related mutations are also associated with reduced phosphorylation of LRRK2, particularly in a cluster of serines (binding site P14 3 3), and are related to a change in cell localization of the protein which varies as a function of cell type and which is also found altered in PD patients. Phosphorylation sites are also related to the kinase activity of the protein because they are dephosphorylated in the presence of LRRK2 inhibitors. The serine/threonine protein phosphatase 1 (PP1) is responsible for LRRK2 dephosphorylation observed in PD mutant LRRK2 and after LRRK2 kinase inhibition.

Inventors identified the interacting region of LRRK2 with PP1. They have been able to design a specific family of peptides that, by inhibiting PP1-LRRK2 interaction provides valuable candidates in treating neurological disorders. There is no disclosure in the art of such inhibitors of LRRK2/PP1 interaction, nor of their use in the treatment of neurological disorders, even less in the treatment of α-synucleinopathies which yet remain an unmet medical need.

### SUMMARY OF THE INVENTION

Invention is thus related to new peptides that constitute valuable candidates for treating neurological disorders. Indeed, besides the current knowledge in the art of the role of the impact of mutations within LRRK2 protein in neurological processes, herein provided experimental data show that, in vitro, peptides of the invention are able to compete with LRRK2/PP1 interaction, and, in cellulo, are internalized within cells thereby exhibiting biological effects as, for example in neuronal cells, an improvement in neurite outgrowth.

In one aspect, the invention thus relates to a peptide which consists of a fragment of polypeptide of SEQ ID NO:1 or variant thereof and which comprises at least the 7 consecutive amino acids ranging from amino acid residue at position 1709 to amino acid at position 1715 of said SEQ ID NO:1 or variant thereof.

In further optional aspects said peptide:
comprises 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50; or consecutive amino acids in SEQ ID NO:1 or variant thereof,
is 18 amino acids long,
consists of a sequence of amino acids in the region ranging from the residue at position 1701 to the amino acid residue at position 1718 of SEQ ID NO:1 or variant thereof, optionally having 60% of identity with the sequence of said region,
consists of a sequence of amino acids in the region ranging from the residue at position 1703 to the amino acid residue at position 1715 of SEQ ID NO:1 or variant thereof, optionally having 60% of identity with the sequence of said region,
consists of a sequence having at least 70% of identity with the sequence of at least 7 amino acids ranging from the amino acid residue at position 1709 to the amino acid residue at position 1715 in SEQ ID NO:1, or variant thereof,
comprises the amino acid residues W1705, S1706, R1707, I1709, R1711, L1712, L1713 and E1714 of SEQ ID NO:1.

In another particular aspect said peptide of the invention is fused to a carrier (or vectorization) peptide in order to allowing the proper targeting of the peptide of the invention in a specifically targeted body compartment and/or cell type and/or subcellular compartment, for example of sequence VKKKKIKAEIKI (SEQ ID NO: 29) or THRPPMWSPVWP (SEQ ID NO: 30).

In another particular aspect, said peptide is selected from peptides of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14 or variants thereof.

In another particular aspect, the invention relates to a nucleic acid molecule encoding for the peptide of the invention and in furthers aspects, to a vector which comprises said nucleic acid molecule or a host cell transformed with said nucleic acid or vector.

Peptides of the invention result from the identification by the inventors of the region of interaction between PP1 and LRRK2, and the design of peptides able to efficiently inhibiting said interaction. Consequently, as mentioned, in one aspect invention relates to said peptides. In another aspect, invention relates to agents directed against this region or peptides as antibodies and/or aptamers. Further in an aspect the invention relates to an inhibitor of LRRK2/PP1 interaction which consists of said peptides or variants thereof.

As shown in the experimental section, peptides of the invention provide a significant improvement in neurites outgrowth when internalized in neuronal cells, which is of particular interest for treating neurodegenerative disorders in which neurite outgrowth and synaptic plasticity are of the first impaired neuronal processes in such diseases.

Consequently, in one aspect the invention is related to the above-mentioned polypeptides, nucleic acids, aptamers or antibodies, which impair LRRK2/PP1 interaction, for use as a medicament. In a particular aspect, the invention relates to an inhibitor of LRRK2/PP1 interaction which consists of said peptides or variants thereof for use as a medicament.

In a more particular aspect, the invention is related to the above-mentioned polypeptides, nucleic acids, aptamer or antibody, which impair LRRK2/PP1 interaction for use in the treatment of a neurodegenerative disorder and in a further aspect said neurodegenerative disorder is an α-synucleinopathy, preferably selected from Parkinson's disease (PD), dementia with Lewy bodies (DLB), and multiple system atrophy (MSA). In a particular aspect, the invention relates to an inhibitor of LRRK2/PP1 interaction which consists of said peptides or variants thereof for use in the treatment of a neurodegenerative disorder, preferably an α-synucleinopathy, more preferably selected from Parkinson's disease (PD), dementia with Lewy bodies (DLB), and multiple system atrophy (MSA).

Other advantages and features of the invention will appear in the description below, which is given herein solely in an illustrative purpose and in a non-limitative way.

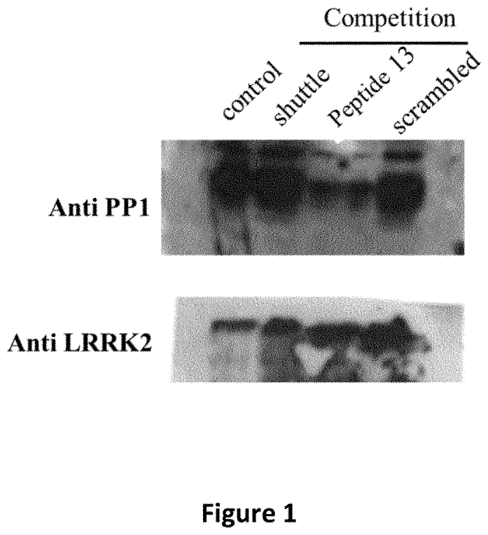

FIG. 1: In vitro competition assay of LRRK2/PP1 interaction with peptide according to the invention. Western blotting using PP1 antibody shows an important decrease in PP1 detected in LRRK2 immunoprecipitates after incubation with peptide 13. Conversely, no difference is noticed when immunoprecipitates are incubated with either shuttle peptide VKKKKIKAEIKI (SEQ ID NO: 29) (“shuttle”) or scramble peptide 7 fused to shuttle peptide (“scrambled”). Control: no competed immunoprecipitates. Immunoprecipitates were washed and immunoblotted with anti-PP1 and anti-LRRK2 antibody.

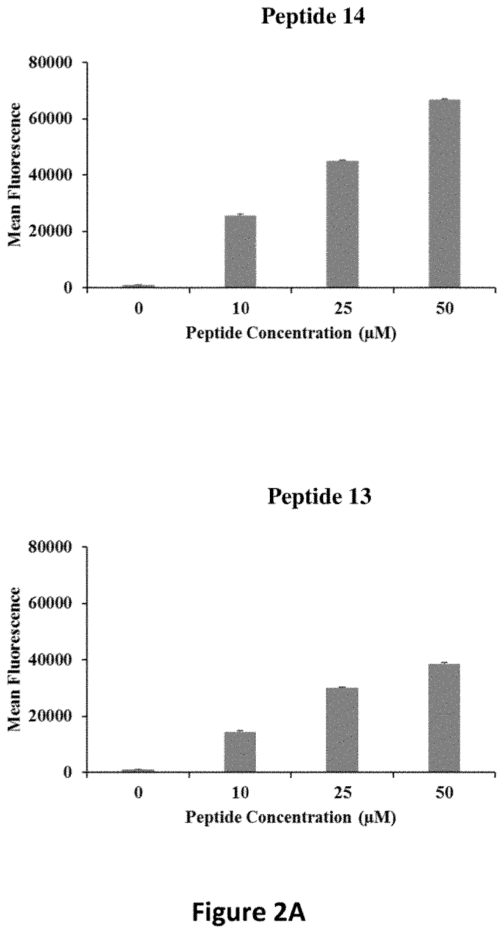

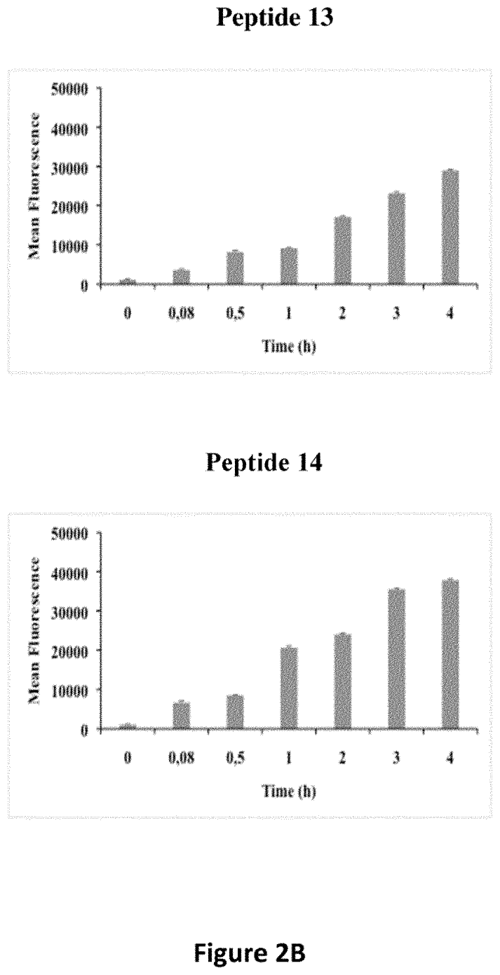

FIG. 2: Internalization of FITC labelled peptides of the invention in MDA-MB231 cell line-study. A. as a function of peptide concentration B. as a function of time of incubation of cell with a composition comprising peptides of the invention. Results show a peptide concentration and incubation time dependency for internalization of peptides of the invention.

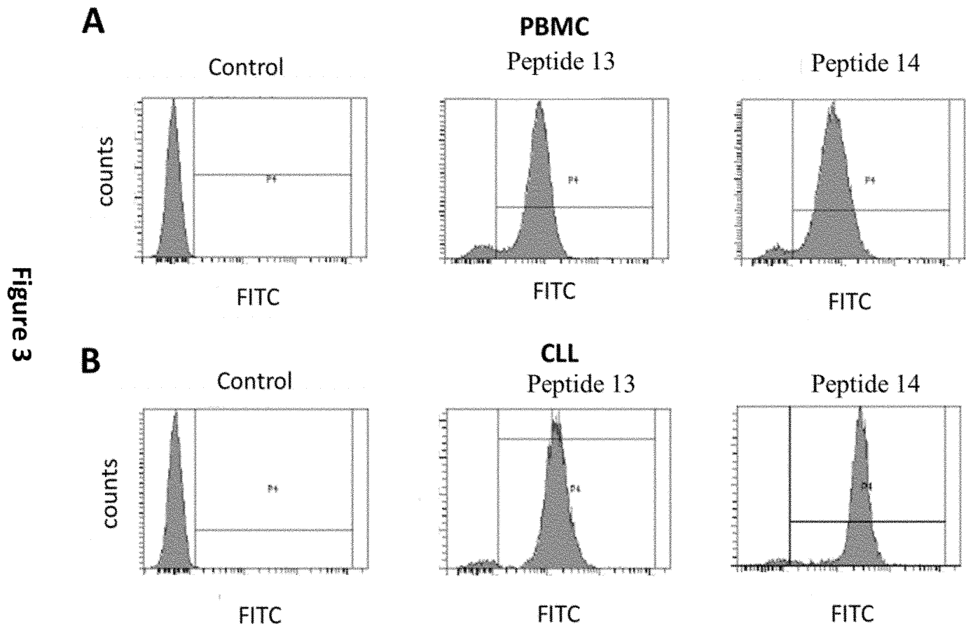

FIG. 3: Internalization of FITC labelled peptides of the invention in primary cells. A. in Peripheral Blood Mononuclear Cells (PBMC) of healthy human. B. in PBMC of chronic lymphocytic leukemia (CLL). FACS analysis: No FITC containing cells is detected in control samples (“control”: control cells, not incubated with any peptide), whereas a majority of FITC labelled cells is detected for cell samples incubated with either of FITC labelled peptide 13 or 14.

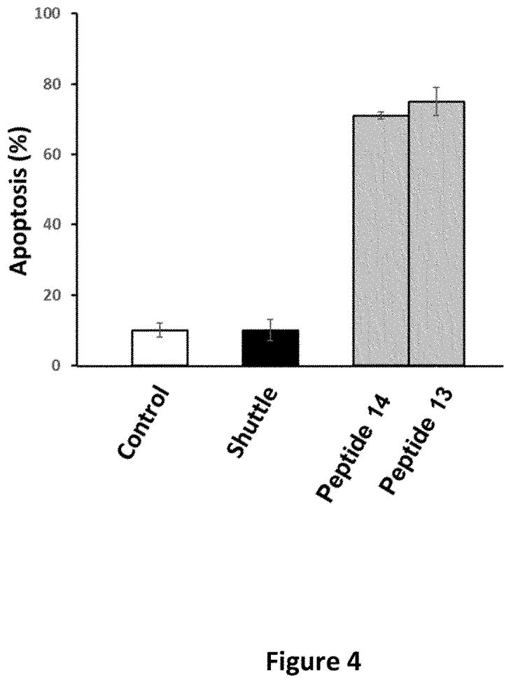

FIG. 4: Apoptosis induction upon internalisation of peptides of the invention in cancer cells. Upon 24 h of treatment with 25 μM of peptide 13 or 14 a strong level of apoptosis of MDA-MB231 cells (grey bars) is noticed while non-treated cells (control, white bar) or cells incubated with only the shuttle peptide (shuttle, black bar) do not show apoptosis.

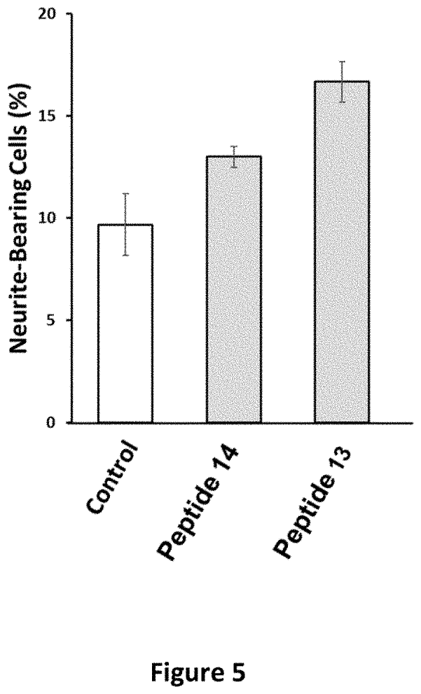

FIG. 5: Induction of neurite outgrowth upon internalisation of peptides of the invention in neuronal cells. An increase in neurite formation is observed in living PC12 neuronal cell line upon incubation with peptides of the invention (grey bars) when compared with non-treated cells (control, white bar).

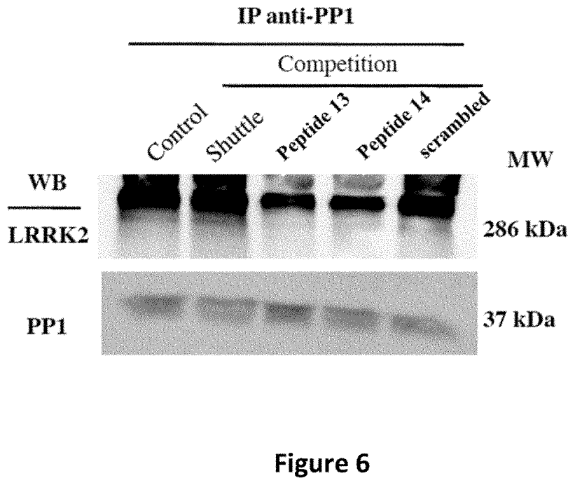

FIG. 6: In vitro competition assay of LRRK2/PP1 interaction with peptides according to the invention. Western blotting using PP1 antibody shows an important decrease in PP1 detected in LRRK2 immunoprecipitates after incubation with peptides 13 or 14. Conversely, no difference is noticed when immunoprecipitates are incubated with either shuttle peptide VKKKKIKAEIKI (SEQ ID NO: 29) (“shuttle”) or scramble peptide 7 fused to shuttle peptide (“scrambled”). Control: no competed immunoprecipitates. Immunoprecipitates were washed and immunoblotted with anti-PP1 and anti-LRRK2 antibody

### DETAILED DESCRIPTION OF THE INVENTION

In the present invention, the inventors investigated the interaction of PP1 and LRRK2. The inventors identified the molecular binding site of LRRK2 with PP1. The inventors further identify the polypeptide region of LRRK2, which when produced in isolation is able to inhibit PP1/LRRK2 interaction thereby providing unexpected and valuable biological effects as shown in the experimental section.

Polypeptides of the Invention

The present invention relates to isolated, synthetic or recombinant polypeptides which are inhibitors of LRRK2/PP1 interaction.

As used herein the term “LRRK2” has its general meaning in the art and refers to Leucine-rich repeat kinase 2, also known as dardarin is an enzyme that in humans is encoded by the PARK8 gene. LRRK2 is a member of the leucine-rich repeat kinase family. Variants of this gene are associated with an increased risk of Parkinson's disease and also Crohn's disease. An exemplary human polypeptide sequence of LRRK2 is SEQ ID NO: 1. Of course, based on the teachings of this disclosure, the skilled in the art is able to identify within the amino acid sequence of homologous protein to LRRK2 of mammals the corresponding region of interaction of LRRK2 and PP1.

SEQ ID NO: 1
MASGSCQGCEEDEETLKKLIVRLNNVQEGKQIETLVQILEDLLVFTYSEHA
SKLFQGKNIHVPLLIVLDSYMRVASVQQVGWSLLCKLIEVCPGTMQSLMGP
QDVGNDWEVLGVHQLILKMLTVHNASVNLSVIGLKTLDLLLTSGKITLLIL
DEESDIFMLIFDAMHSFPANDEVQKLGCKALHVLFERVSEEQLTEFVENKD
YMILLSASTNFKDEEEIVLHVLHCLHSLAIPCNNVEVLMSGNVRCYNIVVE
AMKAFPMSERIQEVSCCLLHRLTLGNFFNILVLNEVHEFVVKAVQQYPENA
ALQISALSCLALLTETIFLNQDLEEKNENQENDDEGEEDKLFWLEACYKAL
TWHRKNKHVQEAACWALNNLLMYQNSLHEKIGDEDGHFPAHREVMLSMLMH
SSSKEVFQASANALSTLLEQNVNFRKILLSKGIHLNVLELMQKHIHSPEVA
ESGCKMLNHLFEGSNTSLDIMAAVVPKILTVMKRHETSLPVQLEALRAILH
FIVPGMPEESREDTEFHHKLNMVKKQCFKNDIHKLVLAALNRFIGNPGIQK
CGLKVISSIVHFPDALEMLSLEGAMDSVLHTLQMYPDDQEIQCLGLSLIGY
LITKKNVFIGTGHLLAKILVSSLYRFKDVAEIQTKGFQTILAILKLSASFS
KLLVHHSFDLVIFHQMSSNIMEQKDQQFLNLCCKCFAKVAMDDYLKNVMLE
RACDQNNSIMVECLLLLGADANQAKEGSSLICQVCEKESSPKLVELLLNSG
SREQDVRKALTISIGKGDSQIISLLLRRLALDVANNSICLGGFCIGKVEPS
WLGPLFPDKTSNLRKQTNIASTLARMVIRYQMKSAVEEGTASGSDGNFSED
VLSKFDEWTFIPDSSMDSVFAQSDDLDSEGSEGSFLVKKKSNSISVGEFYR
DAVLQRCSPNLQRHSNSLGPIFDHEDLLKRKRKILSSDDSLRSSKLQSHMR
HSDSISSLASEREYITSLDLSANELRDIDALSQKCCISVHLEHLEKLELHQ
NALTSFPQQLCETLKSLTHLDLHSNKFTSFPSYLLKMSCIANLDVSRNDIG
PSVVLDPTVKCPTLKQFNLSYNQLSFVPENLTDVVEKLEQLILEGNKISGI
CSPLRLKELKILNLSKNHISSLSENFLEACPKVESFSARMNFLAAMPFLPP
SMTILKLSQNKFSCIPEAILNLPHLRSLDMSSNDIQYLPGPAHWKSLNLRE
LLFSHNQISILDLSEKAYLWSRVEKLHLSHNKLKEIPPEIGCLENLTSLDV
SYNLELRSFPNEMGKLSKIWDLPLDELHLNFDFKHIGCKAKDIIRFLQQRL
KKAVPYNRMKLMIVGNTGSGKTTLLQQLMKTKKSDLGMQSATVGIDVKDWP
IQIRDKRKRDLVLNVWDFAGREEFYSTHPHFMTQRALYLAVYDLSKGQAEV
DAMKPWLFNIKARASSSPVILVGTHLDVSDEKQRKACMSKITKELLNKRGF
PAIRDYHFVNATEESDALAKLRKTIINESLNFKIRDQLVVGQLIPDCYVEL
EKIILSERKNVPIEFPVIDRKRLLQLVRENQLQLDENELPHAVHFLNESGV
LLHFQDPALQLSDLYFVEPKWLCKIMAQILTVKVEGCPKHPKGIISRRDVE
KFLSKKRKFPKNYMSQYFKLLEKFQIALPIGEEYLLVPSSLSDHRPVIELP
HCENSEIIIRLYEMPYFPMGFWSRLINRLLEISPYMLSGRERALRPNRMYW
RQGIYLNWSPEAYCLVGSEVLDNHPESFLKITVPSCRKGCILLGQVVDHID
SLMEEWFPGLLEIDICGEGETLLKKWALYSFNDGEEHQKILLDDLMKKAEE
GDLLVNPDQPRLTIPISQIAPDLILADLPRNIMLNNDELEFEQAPEFLLGD
GSFGSVYRAAYEGEEVAVKIFNKHTSLRLLRQELVVLCHLHHPSLISLLAA
GIRPRMLVMELASKGSLDRLLQQDKASLTRTLQHRIALHVADGLRYLHSAM
IIYRDLKPHNVLLFTLYPNAAIIAKIADYGIAQYCCRMGIKTSEGTPGFRA
PEVARGNVIYNQQADVYSFGLLLYDILTTGGRIVEGLKFPNEFDELEIQGK
LPDPVKEYGCAPWPMVEKLIKQCLKENPQERPTSAQVFDILNSAELVCLTR
RILLPKNVIVECMVATHHNSRNASIWLGCGHTDRGQLSFLDLNTEGYTSEE
VADSRILCLALVHLPVEKESWIVSGTQSGTLLVINTEDGKKRHTLEKMTDS
VTCLYCNSFSKQSKQKNFLLVGTADGKLAIFEDKTVKLKGAAPLKILNIGN
VSTPLMCLSESTNSTERNVMWGGCGTKIFSFSNDFTIQKLIETRTSQLFSY
AAFSDSNIITVVVDTALYIAKQNSPVVEVWDKKTEKLCGLIDCVHFLREVM
VKENKESKHKMSYSGRVKTLCLQKNTALWIGTGGGHILLLDLSTRRLIRVI
YNFCNSVRVMMTAQLGSLKNVMLVLGYNRKNTEGTQKQKEIQSCLTVWDIN
LPHEVQNLEKHIEVRKELAEKMRRTSVE.

As used herein the term “PP1” for Protein Phosphatase 1 has its general meaning in the art, that is serine/threonine protein phosphatase 1. PP1 is one of the most ubiquitous and abundant serine/threonine phosphatases in eukaryotic cells. Protein Phosphatases are implicated in the regulation of various essential cellular functions: PP1 is found to play pivotal role in a wide variety of physiological and molecular processes as glycogen metabolism, muscle contraction, cell progression, neuronal activities, apoptosis etc. . . . and consequently, suspected to implicated in numerous complex diseases. This versatility can be explained by the nearly 200 validated interactors in vertebrates (among which LRRK2).

As used herein the terms “polypeptide” and “peptide” are used interchangeably. They designate a chain of amino acid monomers linked by peptides bonds. Of course, in some instance, specifically when using analogs to some amino-acids which are commonly used in peptide derivative pharmaceutical treatments, said bond can be different from the usual peptide bond. Also, said peptide or polypeptide can be linear or cyclic. In some embodiment peptide of the invention is cyclic as cyclic peptides tend to be extremely resistant to the processes of degradation which is of particular interest for the sake of developing medicaments.

As used herein the term “fragment” of peptide of SEQ ID NO:1 correspond to a polypeptide that is substantially shorter than the whole LRRK2 protein of sequence SEQ ID NO:1 and that consequently is devoid of any the enzymatic activities of said protein. Typically said fragment is a stretch of 7, 8, 9, 10, 11, 12, 13, 14; 15, 16, 17, 18, 19, 20, 21, 22, 23, 24, 25, 26, 27, 28, 29, 30, 31, 32, 33, 34, 35, 36; 37, 38, 39, 40, 41, 42, 43, 44, 45, 46, 47, 48, 49, or 50 consecutive amino acids from SEQ ID NO:1 or variant thereof.

As used herein the term “inhibitor of LRRK2/PP1 interaction” refers to a compound that is able to inhibit the interaction of LRRK2 with PP1. As demonstrated in the example section, polypeptides of the invention are able to inhibit LRRK2/PP1 interaction. Based on structural analysis of this region, Inventors have been able to delineate the “minimum” peptide, and also, minimum identity percentage within this region of interest, by comparing the human sequence of the LRRK2 domain containing the peptide sequence with the complete set of sequences of the UniProt repository (December 2018) using blastp (website: //blast.ncbi.nlm.nih.gov/Blast.cgi?PAGE=Proteins) with an E value of 10, and selecting sequences aligned with that of the peptide using clustal omega. The invention makes thus use of peptides inhibitors of LRRK2/PP1 interaction consisting in a fragment of sequence SEQ ID No 1 as exposed therein or a variant thereof.

The variants include, for instance, naturally-occurring variants due to allelic variations between individuals (e.g., polymorphisms). The term variant also includes LRRK2 sequences from other sources or organisms. Variants are preferably substantially homologous to peptides inhibitors of LRRK2/PP1 interaction consisting in a fragment of sequence SEQ ID No 1 as exposed therein, i.e., exhibit a nucleotide sequence identity of typically at least about 60%, preferably at least about 70%, more preferably at least about 90%, more preferably at least about 95% with sequence of peptides inhibitors of LRRK2/PP1 interaction consisting in a fragment of sequence SEQ ID No 1 as exposed therein.

In one embodiment said inhibitor of LRRK2/PP1 interaction of the invention is a polypeptide which consists of a fragment of peptide of SEQ ID NO:1 and which comprises at least the 7 consecutive amino acids ranging from amino acid residue at position 1709 to amino acid at position 1715 of said SEQ ID NO:1. In a more particular embodiment said polypeptide has at least 70% of identity with the sequence of at least 7 amino acids ranging from the amino acid residue at position 1709 to the amino acid residue at position 1715. In another particular embodiment, said polypeptide which consists of a fragment of peptide of SEQ ID NO:1 and which comprises at least the 7 consecutive amino acids ranging from amino acid residue at position 1709 to amino acid at position 1715 of said SEQ ID NO:1, further comprises the amino acid residues W1705, S1706, R1707, I1709, R1711, L1712, L1713, E1714.

In another embodiment the above mentioned peptide, which is a fragment of peptide of SEQ ID NO:1 and which comprises at least the 7 consecutive amino acids ranging from amino acid residue at position 1709 to amino acid at position 1715 of said SEQ ID NO:1, comprises 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 consecutive amino acids in SEQ ID NO:1. In more particular embodiment said peptide is 18 amino acids long.

In another embodiment, a polypeptide of the invention, which is fragment of peptide of SEQ ID NO:1 and which comprises at least the 7 consecutive amino acids ranging from amino acid residue at position 1709 to amino acid at position 1715 of said SEQ ID NO:1, consists of a sequence of amino acids in the region ranging from the residue at position 1701 to the amino acid residue at position 1718 of SEQ ID NO:1. In more particular embodiment, said polypeptide display at least 60% of identity with the sequence which ranges from the amino acid residue at position 1701 to the amino acid residue at position 1718 in SEQ ID NO:1.

In another embodiment, a polypeptide of the invention consists of a sequence of amino acids in the region ranging from the residue at position 1703 to the amino acid residue at position 1715. In a more particular embodiment said polypeptide consists of the sequence having at least 60% of identity with the sequence which ranges from the amino acid residue position 1703 to the amino acid residue at position 1715 in SEQ ID NO:1.

In another embodiment a polypeptide of the invention consists of a sequence of at least 7 amino acids in the region ranging from the residue at position 1701 to the amino acid residue at position 1718 of SEQ ID NO:1. In more particular embodiments a polypeptide of the invention consists of a sequence of 8, 9, 10, 11, 12, 13, 14, 15, 16, 17 or even 18 amino acids in the region ranging from the residue at position 1701 to the amino acid residue at position 1718 of SEQ ID NO:1.

In another embodiment said peptide inhibitor of LRRK2/PP1 interaction is selected from:

SEQ ID NO: 2:
INRLLEISPY,
SEQ ID NO: 3:
LINRLLEISPY,
SEQ ID NO: 4:
SRLINRLLEISPY,
SEQ ID NO: 5:
PMGFWSRLI,
SEQ ID NO: 6:
GFWSRLINRLLEISPY,
SEQ ID NO: 7:
PMGFWSRLINRLLEISPY,
SEQ ID NO: 8:
GFWSRLINRLLEI,
and
SEQ ID NO: 9:
PMGFWSRLINRLLEI.

In order of allowing the proper targeting of the peptides of the invention in a specifically targeted body compartment and/or cell type and/or subcellular compartment, peptides of the invention can be fused to dedicated peptides. Those vectorization (or carrier) peptides can be for example anyone of those disclosed in patent application WO/2016/156536 which are able to properly vectorize peptides within cells, for example, VKKKKIKREIKI (SEQ ID NO: 31), VKKKKIKAEIKI (SEQ ID NO: 29), VKKKKIKKEIKI (SEQ ID NO: 32) or VKKKKIKNEIKI (SEQ ID NO: 33). Vectorization peptides of interest can also be those which allow delivery of the peptides of the invention across the Blood Brain Barrier (BBB), as those disclosed in patent application WO/2015/001015 and by Prades et al. (2015) or in table 1 of Oller-Salvia et al. (2016), as for example THRPPMWSPVWP (SEQ ID NO: 30). All those peptides are incorporated herein by reference. Of course, peptides of the invention can be targeted in the desired body compartment and/or cell of body through any mean that the skilled in the art could consider, e.g., inter alia nanoparticles, as detailed, for example in “Peptide and Protein Delivery” (Academic press, 2011).

Agents Related to the Peptides of the Invention

In an embodiment the invention relates to a nucleic acid sequence encoding for a polypeptide inhibitor of LRRK2/PP1 interaction according to the invention as described previously.

As used herein, a sequence “encoding” an expression product, such as a RNA, polypeptide, protein, or enzyme, is a nucleotide sequence that, when expressed, results in the production of that RNA or corresponding polypeptide i.e., the nucleotide sequence encodes an amino acid sequence for that polypeptide. Typically, said nucleic acid is a DNA or RNA molecule, which may be included in a suitable vector, such as a plasmid, cosmid, episome, artificial chromosome, phage or viral vector.

So, a further object of the present invention relates to a vector and an expression cassette in which a nucleic acid molecule encoding for a polypeptide of the invention is associated with suitable elements for controlling transcription (in particular promoter, enhancer and, optionally, terminator) and, optionally translation, and also the recombinant vectors into which a nucleic acid molecule in accordance with the invention is inserted. These recombinant vectors may, for example, be cloning vectors, or expression vectors.

As used herein, the terms “vector”, “cloning vector” and “expression vector” mean the vehicle by which a DNA or RNA sequence encoding a peptide according to the invention can be introduced into a host cell, so as to transform the host and promote expression (e.g. transcription and translation) of the introduced sequence.

Any expression vector for animal cell can be used and will be easily identified by the skilled in the art.

A further aspect of the invention relates to a host cell comprising a nucleic acid molecule encoding for a polypeptide according to the invention or a vector according to the invention. These host cells can be used for example to produce peptides of the invention, to amplify genetic material or for gene therapy purposes. In particular, a subject of the present invention is a prokaryotic or eukaryotic host cell genetically transformed with at least one nucleic acid molecule or vector according to the invention.

The construction of expression vectors in accordance with the invention, and the transformation of the host cells can be carried out using conventional molecular biology techniques well known from those skilled in the art.

More particularly, nucleic acid sequences encoding said peptides can be delivered into cells of interest by any suitable mean, in e.g., inter alia viral vectors, in order to allow the expression of said peptides into targeted cells thereby rendering useless fusion to vectorization peptides. Gene delivery viral vectors useful in the practice of the present invention can be constructed utilizing methodologies well known in the art of molecular biology. Typically, viral vectors carrying transgenes (in other words gene encoding peptide of the invention) are assembled from polynucleotides encoding the transgene, suitable regulatory elements and elements necessary for production of viral proteins which mediate cell transduction.

The terms “Gene transfer” or “gene delivery” refer to methods or systems for reliably inserting foreign DNA into host cells. Such methods can result in transient expression of non-integrated transferred DNA, extrachromosomal replication and expression of transferred replicons (e. g., episomes), or integration of transferred genetic material into the genomic DNA of host cells.

Examples of viral vector include adenoviral, retroviral, herpesvirus and adeno-associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a preferred embodiment, adeno-associated viral (AAV) vectors are employed. By an “AAV vector” is meant a vector derived from an adeno-associated virus serotype, including without limitation, AAV-1, AAV-2, AAV-3, AAV-4, AAV-5, AAV6, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g., by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. encoding a peptide of the invention) and a transcriptional termination region.

The control elements are selected to be functional in a mammalian cell. The resulting construct which contains the operatively linked components is bounded (5′ and 3′) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e. g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e. g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-6, etc. Furthermore, 5′ and 3′ITRs which flank a selected nucleotide sequence in an AAV vector need not necessarily be identical or derived from the same AAV serotype or isolate, so long as they function as intended, i. e. to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV-5, AAV-6, etc. Furthermore, 5′ and 3′ ITRs which flank a selected nucleotide sequence in an AAV expression vector need not necessarily be identical or derived from the same AAV serotype or isolate, so long as they function as intended, i. e. to allow for excision and rescue of the sequence of interest from a host cell genome or vector, and to allow integration of the DNA molecule into the recipient cell genome when AAV Rep gene products are present in the cell.

Particularly preferred are vectors derived from AAV serotypes having tropism for and high transduction efficiencies in cells of the mammalian CNS, particularly neurons. A review and comparison of transduction efficiencies of different serotypes is provided in Davidson et al., 2000. In one preferred example, AAV-2 based vectors have been shown to direct long-term expression of transgenes in CNS, preferably transducing neurons. In other nonlimiting examples, preferred vectors include vectors derived from AAV-4 and AAV-5 serotypes, which have also been shown to transduce cells of the CNS (Davidson et al., supra).

The selected nucleotide sequence is operably linked to control elements that direct the transcription or expression thereof in the subject in vivo. Such control elements can comprise control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PKG) promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), Rous sarcoma virus (RSV) promoter, synthetic promoters, hybrid promoters, and the like. In addition, sequences derived from non-viral genes, such as the murine metallothionein gene, will also find use herein. Such promoter sequences are commercially available from, e. g., Stratagene (San Diego, Calif.). For purposes of the present invention, both heterologous promoters and other control elements, such as CNS-specific and inducible promoters, enhancers and the like, will be of particular use.

Examples of heterologous promoters include the CMV promoter. Examples of CNS specific promoters include those isolated from the genes from myelin basic protein (MBP), glial fibrillary acid protein (GFAP), and neuron specific enolase (NSE).

Examples of inducible promoters include DNA responsive elements for ecdysone, tetracycline, etc.

The AAV expression vector which harbors the DNA molecule encoding the peptides of the invention bounded by AAV ITRs, can be constructed by directly inserting the selected sequence (s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e. g., U.S. Pat. Nos. 5,173,414 and 5,139,941; International patent applications WO 92/01070 and WO 93/03769; Lebkowski et al., 1988; Vincent et al., 1990; Carter, 1992; Muzyczka, 1992; Kotin, 1994; Shelling and Smith, 1994; and Zhou et al., 1994.) Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e. g., U.S. Pat. No. 5,139,941. In particular, several AAV vectors are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. In order to produce AAV virions, an AAV expression vector is introduced into a suitable host cell using known techniques, such as by transfection. A number of transfection techniques are generally known in the art. See, e. g. Sambrook et al. (1989): Molecular Cloning, a laboratory manual, Cold Spring Harbor Laboratories, New York; Davis et al. (1986): Basic Methods in Molecular Biology, Elsevier, and Chu et al., 1981. Particularly suitable transfection methods include calcium phosphate co-precipitation, direct microinjection into cultured cells, electroporation, liposome mediated gene transfer, lipid-mediated transduction and nucleic acid delivery using high-velocity microprojectiles.

An “Inhibitor of LRRK2/PP1 interaction” according to the invention can also be an agent that, through its binding to the region ranging from the amino acid residue at position 1701 to the amino acid at position 1718 in SEQ ID NO: I or to any of the peptides described previously, impedes a proper LRRK2/PP1 interaction. Said compound can be, for example, an aptamer or an antibody.

The terms “compound” or “agent” are used herein indifferently to designate the peptides or agents inhibitors of LRRK2/PP1 interaction.

Consequently, in some embodiments, the aptamer or antibody of the present invention specifically bind to a peptide consisting of a fragment of peptide of SEQ ID NO:1 and which comprises at least the 7 consecutive amino acids ranging from amino acid residue at position 1709 to amino acid at position 1715 of said SEQ ID NO:1 as defined above, thereby resulting inhibiting LRRK2/PP1 interaction. In some embodiments, the aptamer or antibody of the present invention specifically binds to one of the polypeptides as described in any preceding paragraphs [0023] to [0032].

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404097 and WO 93/11 161, whereas linear antibodies are further described in Zapata et al. (1995). Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

In natural antibodies, two heavy chains are linked to each other by disulfide bonds and each heavy chain is linked to a light chain by a disulfide bond. There are two types of light chain, lambda (1) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)₂” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab” refers to an antibody fragment having a molecular weight of about 50,000 and antigen binding activity, which is obtained by cutting a disulfide bond of the hinge region of the F(ab′)₂.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)₂.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Monoclonal antibodies may be generated using the method of Kohler and Milstein (1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. polypeptides of the present invention). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant polypeptide of the invention may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In some embodiments, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGI, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., I. Mol. Biol. 294: 151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′)₂ Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDRI and/or CDR2 and/or light chain CDR3 regions have been replaced by homologous human or non-human sequences; chimeric Fab fragment antibodies in which the FR and/or CDRI and/or CDR2 and/or light chain CDR3 regions have been replaced by homologous human or non-human sequences; and chimeric Fd fragment antibodies in which the FR and/or CDRI and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGI, IgG2, IgG3 and IgG4.

Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by Exponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods (Colas et al., 1996).

### Therapeutic Methods and Uses of the Invention:

As used herein, “treatment” includes the therapy, prevention, prophylaxis, retardation or reduction of symptoms provoked by or of the causes of a disease. When related to a neurodegenerative disease, said neurodegenerative disease is, for example selected from tauopathies like Alzheimer's disease (AD), Frontotemporal dementia Primary age-related tauopathy (PART)/Neurofibrillary tangle-predominant senile dementia, Chronic traumatic encephalopathy (CTE), Progressive supranuclear palsy, Corticobasal degeneration, parkinsonism linked to chromosome 17, Lytico-Bodig disease (Parkinson-dementia complex of Guam), Ganglioglioma and gangliocytoma, Meningioangiomatosis, Postencephalitic parkinsonism, Subacute sclerosing panencephalitis, lead encephalopathy, tuberous sclerosis, Pantothenate kinase-associated neurodegeneration, Fronto temporal dementia and lipofuscinosis. Said neurodegenerative disease is also is selected from α-synucleopathies like Parkinson's disease (PD), dementia with Lewy bodies (DLB), and multiple system atrophy (MSA). In a particular embodiment, said α-synucleopathy is PD.

When related to a neurodegenerative disease, the term “treatment” particularly includes the maintenance or the protection neuronal processes like neurite outgrowth and synaptic plasticity in the treated subjects.

When related to PD, the term “treatment” includes in particular the control of disease progression and associated motor and non-motor symptoms.

LRRK2 is known in the art to be implicated in numerous cellular processes. Peptides of the invention as well as agents “Inhibitors of LRRK2/PP1 interaction” as described above are efficient in altering interaction of LRRK2/PP1 and thereby in altering phosphorylation and localisation of LRRK2. As shown in the experimental section, this results in an improvement of neurite outgrowth and synaptic plasticity for neuronal cells in which LRRK2/PP1 interaction is altered through the peptides and other agents of the invention. This is of particular interest in the frame of the treatment of neurodegenerative diseases, as such neuronal processes are known to be among the first ones altered during the course of these diseases.

In this regard in an embodiment this invention relates to a peptide and/or agent ““Inhibitor of LRRK2/PP1 interaction” of the invention, or salts or prodrugs or derivatives of any purity or sustained release formulations thereof, for use as a medicament.

In a preferred embodiment, this invention relates to a composition comprising at least one peptide and/or agent ““Inhibitor of LRRK2/PP1 interaction” of the invention, or salts or prodrugs or derivatives of any purity or sustained release formulations thereof, for use in the treatment of a neurodegenerative disease. In a more preferred embodiment this invention relates to a composition comprising at least one peptide or agent of the invention, or salts or prodrugs or derivatives of any purity or sustained release formulations thereof, for use in the treatment of a disease selected from tauopathies like Alzheimer's disease (AD), frontotemporal dementia Primary age-related tauopathy (PART)/neurofibrillary tangle-predominant senile dementia, Chronic Traumatic Encephalopathy (CTE), progressive supranuclear palsy, corticobasal degeneration, parkinsonism linked to chromosome 17, Lytico-Bodig disease (Parkinson-dementia complex of Guam), ganglioglioma and gangliocytoma, meningioangiomatosis, postencephalitic parkinsonism, subacute sclerosing panencephalitis, lead encephalopathy, tuberous sclerosis, pantothenate kinase-associated neurodegeneration, fronto temporal dementia and lipofuscinosis. In another preferred embodiment this invention relates to a composition comprising a peptide or agent of the invention, or salts or prodrugs or derivatives of any purity or sustained release formulations thereof, for use in the treatment an α-synucleopathy like Parkinson's disease (PD), dementia with Lewy bodies (DLB), and multiple system atrophy (MSA). In a particularly preferred embodiment, this invention relates to a composition comprising at least one peptide or an agent of the invention, or salts or prodrugs or derivatives of any purity or sustained release formulations thereof, for use in the treatment of PD.

Said at least peptide of said composition is selected among the peptides described above in preceding paragraphs [0023] to [0032].

In a particularly preferred embodiment, the invention relates to a composition comprising at least one peptide selected from:

SEQ ID NO: 2:
INRLLEISPY,
SEQ ID NO: 3:
LINRLLEISPY,
SEQ ID NO: 4:
SRLINRLLEISPY,
SEQ ID NO: 5:
PMGFWSRLI,
SEQ ID NO: 6:
GFWSRLINRLLEISPY,
SEQ ID NO: 7:
PMGFWSRLINRLLEISPY,
SEQ ID NO: 8:
GFWSRLINRLLEI,
or
SEQ ID NO: 9:
PMGFWSRLINRLLEI,

for use in treating a neurodegenerative disease, preferably an α-synucleopathy selected from PD, MSA or DLB. In a more particular embodiment said at least one peptide of said composition is fused to any of vectorization peptide as exposed in paragraph [0033]. In an even more particular embodiment said vectorization peptide is VKKKKIKAEIKI (SEQ ID NO 29). As a consequence in an even more particular embodiment the invention relates to a composition comprising at least one peptide selected from:

SEQ ID NO: 10:
VKKKKIKAEIKINRLLEISPY,
SEQ ID NO: 11:
VKKKKIKAEIKILINRLLEISPY,
SEQ ID NO: 12:
VKKKKIKAEIKISRLINRLLEISPY,
SEQ ID NO: 13:
VKKKKIKAEIKIPMGFWSRLI,
SEQ ID NO: 14:
VKKKKIKAEIKIPMGFWSRLINRLLEISPY,
SEQ ID NO: 15:
VKKKKIKAEIKIGFWSRLINRLLEI,
SEQ ID NO: 16:
VKKKKIKAEIKIGFWSRLINRLLEISPY,
or
SEQ ID NO: 17:
VKKKKIKAEIKIPMGFWSRLINRLLEI,

for use in treating a neurodegenerative disease, preferably an α-synucleopathy selected from PD, MSA or DLB.

In a particularly preferred embodiment said at least one peptide of said composition is fused is fused to a vectorization peptide which allows the delivery of said at least one peptide of the invention across the Blood Brain Barrier (BBB), for example as some of the peptides described in paragraph [0033], for example THR (SEQ ID NO 30). As a consequence in an even more particular embodiment the invention relates to a composition comprising at least one peptide selected from:

SEQ ID NO: 18:
THRPPMWSPVWPINRLLEISPY,
SEQ ID NO: 19:
THRPPMWSPVWPLINRLLEISPY,
SEQ ID NO: 20:
THRPPMWSPVWPSRLINRLLEISPY,
SEQ ID NO: 21:
THRPPMWSPVWPPMGFWSRLI,
SEQ ID NO: 22:
THRPPMWSPVWPGFWSRLINRLLEISPY,
SEQ ID NO: 23:
THRPPMWSPVWPPMGFWSRLINRLLEISPY,
SEQ ID NO: 24:
THRPPMWSPVWPGFWSRLINRLLEI,
SEQ ID NO: 25:
THRPPMWSPVWPMGFWSRLI,
SEQ ID NO: 26:
THRPPMWSPVWPMGFWSRLINRLLEISPY,
SEQ ID NO: 27:
THRPPMWSPVWPMGFWSRLINRLLEI,
SEQ ID NO: 28:
THRPPMWSPVWPPMGFWSRLINRLLEI,

for use in treating a neurodegenerative disease, preferably an α-synucleopathy selected from PD, MSA or DLB.

In another embodiment, a composition of the invention comprises at least one agent inhibitor of LRRK2/PP1 interaction selected from an aptamer or an antibody as described above.

In another embodiment the invention relates to a nucleic acid molecule encoding a peptide inhibitor od LRRK2/PP1 as described herein. In a particular embodiment said nucleic acid encodes one peptide selected from peptide of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO 23, SEQ ID NO 24, SEQ ID NO 25, SEQ ID NO 26, SEQ ID NO 27 or SEQ ID NO 28. In a more particular embodiment, said nucleic acid encoding a peptide inhibitor od LRRK2/PP1 as described herein is within the genome of a viral vector, in particular an adeno-associated viral vector.

In another embodiment the invention relates to a host cell transformed with a nucleic acid molecule encoding a peptide inhibitor od LRRK2/PP1 as described herein. In a particular embodiment said nucleic acid encodes one peptide selected from peptide of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO 23, SEQ ID NO 24, SEQ ID NO 25, SEQ ID NO 26, SEQ ID NO 27 or SEQ ID NO 28.

A composition according to the invention typically comprises one or several pharmaceutically acceptable carriers or excipients. Also, for use in the present invention, compounds of the invention are usually mixed with pharmaceutically acceptable excipients or carriers. In this regard, in a particular embodiment a composition according to the invention is a pharmaceutical composition comprising said peptide and/or agent inhibitor of LRRK2/PP1 interaction as exposed above.

In another preferred embodiment, the invention relates to a method of treating a disease selected from tauopathies like Alzheimer's disease (AD), frontotemporal dementia Primary age-related tauopathy (PART)/neurofibrillary tangle-predominant senile dementia, Chronic Traumatic Encephalopathy (CTE), progressive supranuclear palsy, corticobasal degeneration, parkinsonism linked to chromosome 17, Lytico-Bodig disease (Parkinson-dementia complex of Guam), ganglioglioma and gangliocytoma, meningioangiomatosis, postencephalitic parkinsonism, subacute sclerosing panencephalitis, lead encephalopathy, tuberous sclerosis, pantothenate kinase-associated neurodegeneration, fronto temporal dementia and lipofuscinosis, or α-synucleopathies like Parkinson's disease (PD), dementia with Lewy bodies (DLB), or multiple system atrophy (MSA), comprising administering a peptide and/or an agent inhibitor of LRRK2/PP1 of the invention as exposed above. More particularly, said method comprises administering at least one peptide selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO 23, SEQ ID NO 24, SEQ ID NO 25, SEQ ID NO 26, SEQ ID NO 27 or SEQ ID NO 28.

In a more preferred embodiment, the invention relates to a method of treating PD in a subject in need thereof, comprising administering a peptide and/or an agent inhibitor of LRRK2/PP1 of the invention as exposed above (in the Polypeptides of the invention section). In an even more preferred embodiment, said method comprises administering at least one peptide selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO 23, SEQ ID NO 24, SEQ ID NO 25, SEQ ID NO 26, SEQ ID NO 27 or SEQ ID NO 28.

In a preferred embodiment, the above methods, or compositions can be used in a subject suffering from or who is at risk of developing a neurodegenerative disease as listed above or symptoms associated with said disease.

In a preferred embodiment, the above methods, or compositions can be used in a subject suffering from or who is at risk of developing PD disease or symptoms associated with PD.

The combination of early detection of non-motor symptoms, most particularly anosmia, with imaging techniques (Single-photon emission computed technology, Positron Emission Tomography) to assess changes in striatal dopamine transporter may be a suitable approach to identify at risk PD patients prior to the appearance of motor symptoms, thus allowing early start of neuroprotective therapy using the compounds, compositions or therapies according to the invention.

Some PD cases can be attributed to mutations within genes such as SNCA (alpha-synuclein), PRKN (parkin), LRRK2 (leucine-rich repeat kinase 2), PINK1 (PTEN-induced putative kinase 1), DJ-1 and ATP13A2 and eleven gene loci (PARK1-PARK11). In this regard, in a particular embodiment, the invention relates to the use of the above methods, compositions or therapies for the treatment of PD in a subject having a mutation in at least one of the following genes: SNCA, PRKN, LRRK2, PINK1, DJ-1, ATP13A2 and PARK1 to PARK11.

High concentrations exposure or chronic exposure to metals such as manganese, copper or leads, or chemicals, such as pesticides (e.g. paraquat, rotenone and maneb), are likely to cause PD or related disorders. In this regard, in a particular embodiment, the invention relates to the use of the above methods, compositions or therapies in the treatment of PD or related disorders, in a subject exposed, suspected to have been exposed or at risk of be exposed, to chemicals or metals known to be risk factors for developing PD or related disorders.

The above methods, compositions or therapies may further be used in conjunction or association or combination with additional drugs or treatments.

In a particular embodiment, additional therapies used in conjunction with compositions or compounds for use in treating PD according to the present invention, may comprise one or more drug(s) that ameliorate symptoms of PD, one or more drug(s) that could be used for palliative treatment of PD or one or more drug(s) currently evaluated in the frame of clinical trials for treating of PD. Therefore, compositions of the invention can be combined with dopaminergic drugs such as dopamine precursors (preferably levodopa), dopamine receptor agonists (preferably pergolide, cabergoline, lisuride, pramipexole, ropinirole or apomorphine) or inhibitors of dopamine-metabolizing enzymes (preferably selegiline, rasagiline, tolcapone or entacapone). Compositions of the invention can also be combined with treatment of the non-motor symptoms of PD, preferably Clozapine, Desipramine, Citalopram, Nortriptyline, Paroxetine, Atomoxetine, Venlafaxine, Amantadine, Donepezil, Rivastigmine or Memantine.

In this regard, in a further embodiment this invention relates to a composition, for use in the treatment of PD, comprising a composition as defined above, in combination with at least one compound selected from the group consisting of levodopa, pergolide, cabergoline, lisuride, pramipexole, ropinirole, apomorphine, selegiline, rasagiline, tolcapone, entacapone, clozapine, desipramine, citalopram, nortriptyline, paroxetine, atomoxetine, venlafaxine, amantadine, donepezil, rivastigmine and memantine, or salts or prodrugs or derivatives of any purity or sustained release formulations thereof.

In a particular embodiment, when combination therapies of the invention comprise dopamine precursor, they can be further combined with at least one compound selected from peripheral dopa decarboxylase inhibitors or catechol-O-methyl transferase inhibitors. More particularly, when combination therapies of the invention comprise a dopamine precursor, they can be further combined with at least one compound selected from carbidopa, benserazide or entacapone.

In another embodiment, compositions or combination therapies of the invention can be used in conjunction with surgical therapy for PD such as deep brain stimulation. More particularly, surgical therapies are deep brain stimulation of the subthalamic nucleus or of the globus pallidus interna.

In this regard, the invention also relates to a composition as defined above, for use in combination with deep brain stimulation of the subthalamic nucleus or of the globus pallidus interna, in the treatment of PD and related disorders.

Inventors also show in the experimental part that such peptides are able to induces apoptosis when internalized in cancer cell lines. Also, in a particular embodiment, this invention relates to a composition comprising at least one peptide and/or agent ““Inhibitor of LRRK2/PP1 interaction” of the invention, or salts or prodrugs or derivatives of any purity or sustained release formulations thereof, for use in the treatment of cancer.

Therapy according to the invention may be provided at home, the doctor's office, a clinic, a hospital's outpatient department, or a hospital, so that the doctor can observe the therapy's effects closely and make any adjustments that are needed.

The duration of the therapy depends on the stage of the disease being treated, age and condition of the patient, and how the patient responds to the treatment. When the therapy is combinatorial, the dosage, frequency and mode of administration of each component of the combination can be controlled independently. For example, one compound may be administered orally while the second may be administered intramuscularly. Combination therapy may be given in on-and-off cycles that include rest periods so that the patient's body has a chance to recovery from side-effects. The compounds may also be formulated together such that one administration delivers all compounds.

Possible pharmaceutical compositions include those suitable for oral, rectal, topical (including transdermal, buccal and sublingual), parenteral (including subcutaneous, intramuscular, intravenous and intradermal), or intrathecal administration. More commonly these pharmaceutical formulations are prescribed to the patient in “patient packs” containing a number dosing units or other means for administration of metered unit doses for use during a distinct treatment period in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions. Thus, the invention further includes a pharmaceutical formulation, as herein before described, in combination with packaging material suitable for said formulations. In such a patient pack the intended use of a formulation for the treatment can be inferred by instructions, facilities, provisions, adaptations and/or other means to help using the formulation most suitably for the treatment. Such measures make a patient pack specifically suitable for and adapted for use for treatment with the compounds of the present invention.

The peptide or agent inhibitor of LRRK2/PP1 interaction of the invention may be contained, in any appropriate amount, in any suitable carrier substance. It may be present in an amount of up to 99% by weight of the total weight of the composition. The pharmaceutical composition may be provided in a dosage form that is suitable for the oral, parenteral (e.g., intravenously, intramuscularly), rectal, cutaneous, nasal, vaginal, inhalant, skin (patch), intrathecal or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Typically, the peptide or agent inhibitor of LRRK2/PP1 interaction of the invention of the invention as described above is administered to the subject in a therapeutically effective amount.

By a “therapeutically effective amount” of the peptide or agent inhibitor of LRRK2/PP1 interaction of the present invention as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the agent of the present invention for the symptomatic adjustment of the dosage to the subject to be treated. A medicament typically contains from about 0.01 mg to about 500 mg of the peptide or agent inhibitor of LRRK2/PP1 interaction of the present invention of the present invention, preferably from 1 mg to about 100 mg of the agent of the present invention. An effective amount of the drug is ordinarily supplied at a dosage level from 0.0002 mg/kg to about 20 mg/kg of body weight per day, especially from about 0.001 mg/kg to 7 mg/kg of body weight per day. For the reasons given above, these dosage ranges are intended to provide general guidance and support for the teachings herein, but are not intended to limit the scope of the invention.

The following examples are given for purposes of illustration and not by way of limitation.

### EXAMPLES

### 1. Materials and Methods

Cell Lines

Human cancer breast cell line MDA-MB231 was cultured in DMEM medium supplemented with 10% foetal calf serum (FCS). Peripheral blood mononuclear cells (PBMC) were cultured in RPMI medium supplemented with 10% of FCS, 1% non-essential amino acids, 1% Hepes, 1% sodium pyruvate and 1% glutamine.

Peptides Synthesis and Sequence

Peptides were synthesized in an automated multiple peptide synthesizer with solid phase procedure and standard Fmoc chemistry. The purity and composition of the peptides was confirmed by reverse phase HPLC and mass spectrometry (Frank and Overwin, 1996, Methods in Molecular Biology).

PP1 Binding Assay on Cellulose-Bound Peptides Containing LRRK2 Sequence

Overlapping dodecapeptides with two amino acid shift, spanning the complete LRRK2 sequence were prepared by automatic spot synthesis (Abimed, Langerfeld, Germany) onto an amino-derived cellulose membrane, as described (Frank and Overwin, 1996, Gausepohl et al., 1992). The membrane was saturated using 3% non-fat dry milk/3% BSA (2 h room temperature), incubated with purified PP1 protein (4 μg/ml, 4° C., overnight) and after several washing steps, incubated with polyclonal anti-PP1 antibody 2 h at room temperature, followed by HRP-conjugated secondary antibody for 1 h at room temperature. Positive spots were visualized using the ECL system.

Isolation and Culture of Primary Cells

Fresh blood from healthy donors (HD) was obtained from Etablissement Francais du Sang. Chronic lymphocytic leukemia (CLL) patient samples were obtained from the Department of Hematology. Peripheral blood mononuclear cells (PBMC) from HD and CLL patients were prepared by Ficoll gradient centrifugation. Cells were maintained in RPMI 1640 supplemented with 10% of FCS, 1% non-essential amino acids, 1% Hepes, 1% sodium pyruvate and 1% glutamine. All the experimental protocols as well as the human blood samples isolation were approved by the Ethical Committee of the Hospital in accordance with the National Guide of the Ministry of Health.

Detection of Apoptosis by Annexin-V-FITC Staining

Apoptosis was determined using Annexin-V FITC (eBiosciences) as described by the manufacturer. Briefly, cells were washed in 1× binding buffer, centrifuged and resuspended in 100 μl of 1× binding buffer containing Annexin-V FITC and propidium iodide. After incubation for 15 min, cells were analyzed by flow cytometry. Data acquired by FACS Canto were analyzed with Diva 60 software. Each condition was analyzed in triplicate. The effect of peptide treatment was compared to untreated control cells.

In Vitro Protein/Protein Interaction Competition

The PP1/LRRK2 interaction was competed using the peptides of the invention. Lysates from MDA-MB231 cells were immunoprecipitated with anti-LRRK2 antibody (overnight, 4° C.) and protein A/G Sepharose was added for 1 h at 4° C. The PP1/LRRK2 interaction was competed with 1 mM of the peptide for 30 min at room temperature. After several washing steps, immunoprecipitates were transferred to nitrocellulose and blotted with anti PP1 antibody (Santa Cruz Biotechnology). As internal control, the blot was also blotted with anti-LRRK2 antibody (Invitrogene).

Immunoprecipitation and Western Blotting

Cells (5×10⁶) were lysed for 20 min at 4° C. in lysis buffer (50 mM Tris pH8, 1% NP40, 137 mM NaCl, 1 mM MgCl2, 1 mM CaCl₂), 10% glycerol and protease inhibitor mixture). Lysates (500 μg) were immunoprecipitated with the appropriated antibody overnight at 4° C. and protein A/G sepharose was added for 1 h at 4° C. After washing with 1×TBST (20 mM Tris HCl pH7.5, 150 mM NaCl, 0.05% Tween 20) immunoprecipitates were separated by SDS-PAGE, transferred to nitrocellulose, blocked (5% non-fat dry milk in TBST) and incubated with the primary antibody (Santa Cruz Biotehnology). The membrane was washed and incubated with PO-conjugates secondary antibody (Dako). Protein detection was performed using the ECL system.

Quantification of Cellular Internalization

Human cell line MDA-MB231 was seeded in 24 well plate (1×10⁵ cells/well) and treated with different concentrations of FITC-labelled peptides for different periods of time. After treatment, cells were harvested and washed twice with PBS to remove the extracellular unbound peptide and resuspended in 200 μl of PBS. FITC fluorescence intensity of internalized peptides was measured by flow cytometry. Untreated cells were used as control.

Peptide Internalization Visualization of FITC Labelled Peptides

For intracellular localization of FITC-labelled peptides, MDA-MB231 cells were seeded in a 8 well Labtek (Thermo Fischer). Cells were treated with FITC-labelled peptides for 4 h and fixed with 4% of formaldehyde for 15 min at room temperature. Samples were washed twice with PBS and mounted in mounting buffer. Images were captured with a fluorescence microscopy (Olympus Japan) using 63× magnification objective.

Internalisation in Neuronal Cells and Effects on Neurites Formation

Neuroscreen cell (subclone of rat PC12 neuronal cell) was cultured in 24 well places containing non-differentiating medium at a density of 20.000 cells per well overnight at 37° C. Initial medium was replaced by DF medium containing 10 ng/ml of NGF and 7% of FBS for further 3 h. Cells were treated with control peptide (shuttle alone), peptides of the invention at 0.1 mM in DF medium. Images were recorded over a two days period. Two wells per condition were counted upon 48 h of incubation. Percentage of neurite-bearing cells was monitored at 24 and 48 h after treatment.

Transwell Assay: Ability of the Peptides to Cross the BBB

Transwell inserts made of polyester with 8 mM pore size were used for the assay. Cells were grown until confluence in the upper compartment of a 24-transwell plate. Peptides were put in the upper compartment and the content of the lower compartment was analyzed for detection of peptides by mass spectrometry (MS), and the area of the corresponding pic quantified. MS data were analyzed using the software Cliprot tools, Flex analysis, Bruker.

Analysis of Peptide Integrity on Human Serum

Peptides were incubated at 37° C. in 250 ml of human serum for 1, 3, 5 and 24 hours. Samples were collected and peptide degradation stopped by freezing. Peptides were extracted from samples using the Proteo Miner Protein Enrichment System (Bio-Rad). Percentage of intact peptide was estimated by mass spectrometry (MS) using MALDI-TOFF (Bruker Autoflex II) following supplier protocol. Measurements were performed in triplicate. MS data were analyzed using the software Cliprot tools, Flex analysis, Bruker.

Drosophila Stocks, Culture Methods and Treatment with Compounds

Drosophila melanogaster lines models for PD were obtained from Bloomington Stock Center. The fly food medium contained 60 g/l yeast extract, 34 g/l cornmeal, 50 g/l sucrose, 14 g/l agarose low gelling temperature and 25 ml/I of methyl 4-hydroxybenzoate. Peptides were incorporated in the food medium at 37° C. at a final concentration ranging from 10 to 100 mM. Untreated control received the same dose of food. After 10 days of development, adult flies were collected and analyzed the modifications in the phenotype. The hG2019S mutant fly model (Liu Z et al. (2008)) is characterized by some features of PD (e.g. loss in TH neurons, absence of response to L DOPA, retinal degeneration, decrease in Locomotor activity).

### 2. Results

Identification of LRRK2 Sequences Involved in PP1 Interaction—In Vitro Interaction Testing for Peptides of the Invention

Overlapping dodecapeptides from LRRK2 protein were generated from LRRK2 protein sequence (SEQ ID NO:1) and immobilized on a cellulose membrane which was then hybridized with PP1 protein as exposed in material and method section. Among the sets of spots inventors selected a one set corresponding to a linear interacting area, spanning the residues 1701 to 1718 and forming a α-helix structure located in an area predicted to be exposed at the surface of the LRRK2 protein. Different peptides have been delineated in order to minimize the entropic cost upon peptide binding.

Chimeric peptides containing VKKKKIKAEIKI (SEQ ID NO: 29) as an N terminal part, an optimized cell penetrating peptide, followed by peptides designed from the interaction sequence of LRRK2 to PP1 identified by the inventors were chemically synthesized (Table 1) and further used for functional analysis. Same has been made with shuttle THR (THRPPMWSPVWP (SEQ ID NO: 30), Oller-Salvia B, 2016), a carrier peptide able to cross the blood brain barrier.

TABLE 1

| | Peptides from LRRK2/PP1 | |
|---|---|---|
| Peptide | interaction domain and fused | |
| number | peptides synthetized | SEQ ID |
| 1 | INRLLEISPY | SEQ ID NO: 2 |
| 2 | LINRLLEISPY | SEQ ID NO: 3 |
| 3 | SRLINRLLEISPY | SEQ ID NO: 4 |
| 4 | PMGFWSRLI | SEQ ID NO: 5 |
| 5 | GFWSRLINRLLEISPY | SEQ ID NO: 6 |
| 6 | PMGFWSRLINRLLEISPY | SEQ ID NO: 7 |
| 7 | GFWSRLINRLLEI | SEQ ID NO: 8 |
| 8 | PMGFWSRLINRLLEI | SEQ ID NO: 9 |
| 9 | VKKKKIKAEIKINRLLEISPY | SEQ ID NO: 10 |
| 10 | VKKKKIKAEIKILINRLLEISPY | SEQ ID NO: 11 |
| 11 | VKKKKIKAEIKISRLINRLLEISPY | SEQ ID NO: 12 |
| 12 | VKKKKIKAEIKIPMGFWSRLI | SEQ ID NO: 13 |
| 13 | VKKKKIKAEIKIPMGFWSRLINRLLEIS | SEQ ID NO: 14 |
| | PY | |
| 14 | VKKKKIKAEIKIGFWSRLINRLLEI | SEQ ID NO: 15 |
| 15 | VKKKKIKAEIKIGFWSRLINRLLEISPY | SEQ ID NO: 16 |
| 16 | VKKKKIKAEIKIPMGFWSRLINRLLEI | SEQ ID NO: 17 |
| 17 | THRPPMWSPVWPINRLLEISPY | SEQ ID NO: 18 |
| 18 | THRPPMWSPVWPLINRLLEISPY | SEQ ID NO: 19 |
| 19 | THRPPMWSPVWPSRLINRLLEISPY | SEQ ID NO: 20 |
| 20 | THRPPMWSPVWPPMGFWSRLI | SEQ ID NO: 21 |
| 21 | THRPPMWSPVWPGFWSRLINRLLEISPY | SEQ ID NO: 22 |
| 22 | THRPPMWSPVWPPMGFWSRLINRLLEIS | SEQ ID NO: 23 |
| | PY | |
| 23 | THRPPMWSPVWPGFWSRLINRLLEI | SEQ ID NO: 24 |
| 24 | THRPPMWSPVWPMGFWSRLI | SEQ ID NO: 25 |
| 25 | THRPPMWSPVWPMGFWSRLINRLLEISPY | SEQ ID NO: 26 |
| 26 | THRPPMWSPVWPMGFWSRLINRLLEI | SEQ ID NO: 27 |
| 27 | THRPPMWSPVWPPMGFWSRLINRLLEI | SEQ ID NO: 28 |

Said peptides were then tested to their ability to target the in vitro interaction LRRK2/PP1. This was tested by a competition assay using lysates from MDA-MB231 cell line, that was immunoprecipitated with anti-LRRK2 antibody and the interaction with PP1 was competed using the peptides. As shown in FIG. 1, tested peptides are able to inhibit LRRK2/PP1 interaction: PP1 is detected in control LRRK2 immunoprecipitates and in immunoprecipitates competed using the shuttle peptide alone (VKKKKIKAEI (SEQ ID NO: 29), “shuttle” in FIG. 1) alone or fused to scrambled peptide 6 (corresponding to LRRK2 part of peptide 14), while it was low detected after competition with 1 mM of peptide 13 according to the invention. LRRK2 was used as internal control showing similar level in all conditions. These results show that peptides of the invention specifically target the interaction between human LRRK2 and PP1. A second set of competition assay with peptide 13 and 14 is presented on FIG. 6. Of note, the interaction of PP1 with caspase 9 is not inhibited by peptides of the invention, thereby showing the specificity of their inhibitory activity for LRRK2/PP1 interaction (not shown).

Quantification of Internalization of Peptides of the Invention—Crossing of the BBB

The peptides were labelled with FITC and their internalization was analyzed by FACS. MDA-MB231 cells were treated with FITC-labelled peptides at different concentrations for 4 h and then, internalization analyzed by FACS. FIG. 2A shows the results obtained for peptides 13 and 14 as function of the concentration of peptide which had been used. The fluorescence intensity of internalization is found higher when using peptide 14 (containing the region spanning aa 1703 to 1715 of human LRRK2) compared to when using peptide 13 (containing the region spanning aa 1701 to 1718 of human LRRK2). FIG. 2B compares the kinetic of internalization of two peptides of the invention as a function of time.

Results thus show that structure of peptides of the invention is compatible with their internalization into the cell by using vectorization peptides.

In addition to cell lines, the internalization of the new peptides according to the invention was further tested in peripheral blood mononuclear cells (PBMC) from healthy donors and chronic lymphocytic leukemia (CLL) patients. PBMC from both were incubated with 50 μM of both peptides for 4 h at 37° C. As illustrated on FIG. 3, as for the MDA-MB231 cell line, peptide 14 shows a slightly higher fluorescence intensity than peptide 13 in healthy donors and CLL patients. This result show that, regarding vectorization of peptides of the invention through vectorization peptide, even the longer peptides of the invention are internalized, even if with a slightly less efficacy, in primary cells.

Ability to cross the BBB has been also tested using a carrier peptide as described by Oller-Salvia B (2016), carrier THR (Seq ID NO 30) in a transwell assay using a human brain microvascular EC line as described above. Peptides 22 and 23 were found translocated at levels of same order of magnitude as THR alone (not shown).

Therefore, peptides of the invention can be fused to very different carriers to be addressed in different compartments through very different way as, for example internalization within cell as well as transcytosis (e.g. passage of blood brain barrier). Further peptides have been found rather sable over 24 hours in human serum, which is of particular advantage (not shown) especially for intravenous route of administration and aiming at crossing the BBB.

Effect of Peptides of the Invention on Apoptosis

Given that the complex LRRK2/PP1 plays an important role in the control of several cell functions, peptides of the invention were tested for their capacity to induce apoptosis in human cancer cell lines. As exemplified in FIG. 4, upon 24 h of treatment with 25 μM of peptide 13 and 14, peptides of the invention are able to induce a strong level of apoptosis, while control non-treated cells did not show apoptosis. This functional effect could be mediated by the disruption of the interaction LRRK2/PP1 by the peptides of the invention.

Effect of Peptides of the Invention on Neurites Outgrowth in Neuronal Cells

IncuCyte Live-cell system allows a real-time automated measurement of the dynamic changes of cells of the nervous system. As shown in FIG. 5, an increase (up to 70%) in neurite-bearing cells is observed when neuronal cells are treated with the peptides of the invention when compared to cells treated with the shuttle control peptide.

Treatment of neuronal cells with peptides of the invention therefore results in an improvement in neuronal processes such as neurite outgrowth which are impaired in neurodegenerative diseases.

### 3. Conclusion

Inventors have thus identified the region responsible for the interaction of LRRK2 with PP1. They provide evidences that peptides of the invention are efficient in vitro in disrupting LRRK2/PP1 interaction. Furthermore, inventors show that they can be easily internalized in the targeted cells or cross the BBB by the way, for example of vectorization peptides. Internalization is found to trigger neurite outgrowth on neuronal cells. Peptides of the invention are thus of particular interest in treating neurodegenerative diseases, more particularly α-synucleinopathy, which are known to be linked to LRRK2, and even more particularly in treating PD.

### REFERENCES

Carter B J. Adeno-associated virus vectors. Curr Opin Biotechnol. 1992 October; 3(5):533-9.
Colas P, Cohen B, Jessen T, Grishina I, McCoy J, Brent R. Genetic selection of peptide aptamers that recognize and inhibit cyclin-dependent kinase 2. Nature. 1996 Apr. 11; 380(6574):548-50.
Davidson BL1, Stein C S, Heth J A, Martins I, Kotin R M, Derksen T A, Zabner J, Ghodsi A, Chiorini J A. Recombinant adeno-associated virus type 2, 4, and 5 vectors: transduction of variant cell types and regions in the mammalian central nervous system. Proc Natl Acad Sci USA. 2000 Mar. 28; 97(7):3428-32.
Frank R, Overwin H. SPOT synthesis. Epitope analysis with arrays of synthetic peptides prepared on cellulose membranes. Methods Mol Biol. 1996; 66:149-69.
Gausepohl H, Boulin C, Kraft M, Frank R W. Automated multiple peptide synthesis. Pept Res. 1992 November-December; 5(6):315-20.
Kabat E A, Wu T T. Identical V region amino acid sequences and segments of sequences in antibodies of different specificities. Relative contributions of V H and V L genes, minigenes, and complementarity-determining regions to binding of antibody-combining sites. J Immunol. 1991 Sep. 1; 147(5):1709-19.
Köhler G & Milstein C. Continuous cultures of fused cells secreting antibody of predefined specificity. Nature 1975 256, pages 495-497.
Kotin R M. Prospects for the use of adeno-associated virus as a vector for human gene therapy. Hum Gene Ther. 1994 July; 5(7):793-801.
Lebkowski J S, McNally M M, Okarma T B, Lerch L B. Adeno-associated virus: a vector system for efficient introduction and integration of DNA into a variety of mammalian cell types. Mol Cell Biol. 1988 October; 8(10):3988-96.
Liu Z1, Wang X, Yu Y, Li X, Wang T, Jiang H, Ren Q, Jiao Y, Sawa A, Moran T, Ross C A, Montell C, Smith VWV. A Drosophila model for LRRK2-linked parkinsonism. Proc Natl Acad Sci USA. 2008 Feb. 19; 105(7):2693-8.
MacLeod D, Dowman J, Hammond R, Leete T, Inoue K, Abeliovich A. The familial Parkinsonism gene LRRK2 regulates neurite process morphology. Neuron. 2006 Nov. 22; 52(4):587-93.
Miklossy J, Arai T, Guo J P, Klegeris A, Yu S, McGeer E G, McGeer P L. LRRK2 expression in normal and pathologic human brain and in human cell lines. J Neuropathol Exp Neurol. 2006 October; 65(10):953-63.
Muzyczka N. Use of adeno-associated virus as a general transduction vector for mammalian cells. Curr Top Microbiol Immunol. 1992; 158:97-129.
Oller-Salvia B, Sánchez-Navarro M, Giralt E, Teixido M. Blood-brain barrier shuttle peptides: an emerging paradigm for brain delivery. Chem Soc Rev. 2016 Aug. 22; 45(17):4690-707.
Prades R, Oller-Salvia B, Schwarzmaier S M, Selva J, Moros M, Balbi M, Grazú V, de La Fuente J M, Egea G, Plesnila N, Teixidó M, Giralt E. Applying the retro-enantio approach to obtain a peptide capable of overcoming the blood-brain barrier. Angew Chem Int Ed Engl. 2015 Mar. 23; 54(13):3967-72.
Shelling A N, Smith M G. Targeted integration of transfected and infected adeno-associated virus vectors containing the neomycin resistance gene. Gene Ther. 1994 May; 1:165-9.
Vincent K A, Moore G K, Haigwood N L. Replication and packaging of HIV envelope genes in a novel adeno-associated virus vector system. Vaccines 90: 353-359.
Wu H, Nie Y, Huse W D, Watkins J D Humanization of a murine monoclonal antibody by simultaneous optimization of framework and CDR residues. J Mol Biol. 1999 Nov. 19; 294(1):151-62.
Zapata G, Ridgway J B, Mordenti J, Osaka G, Wong W L, Bennett G L, Carter P. Engineering linear F(ab′)2 fragments for efficient production in Escherichia coli and enhanced antiproliferative activity. Protein Eng. 1995 October; 8(10):1057-62.
Zhou S Z, Cooper S, Kang L Y, Ruggieri L, Heimfeld S, Srivastava A, Broxmeyer H E. Adeno-associated virus 2-mediated high efficiency gene transfer into immature and mature subsets of hematopoietic progenitor cells in human umbilical cord blood. J Exp Med. 1994 Jun. 1; 179(6):1867-75.

### REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “APIC-65011-Sequence-Listing_ST25.txt”, created Aug. 30, 2021, file size of 32,768 bytes, is hereby incorporated by reference.

1. A peptide which consists of a fragment of 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 consecutive amino acids of polypeptide of SEQ ID NO:1 or variant thereof and which comprises at least the 7 consecutive amino acids ranging from amino acid residue at position 1709 to amino acid at position 1715 of said SEQ ID NO:1 or variant thereof.

2. The peptide of claim 1 which is 18 amino acids long.

3. The peptide of claim 1 which consists of a sequence of amino acids in the region ranging from the residue at position 1701 to the amino acid residue at position 1718 of SEQ ID NO:1 or variant thereof.

4. The peptide of claim 1 which consists of a sequence of amino acids in the region ranging from the residue at position 1703 to the amino acid residue at position 1715 of SEQ ID NO:1 or variant thereof.

5. The peptide of claim 1 which consists of a sequence having at least 70% of identity with the sequence of at least 7 amino acids ranging from the amino acid residue at position 1709 to the amino acid residue at position 1715 in SEQ ID NO:1.

6. The peptide of claim 4 which consists of the sequence having at least 60% of identity with the sequence which ranges from the amino acid residue at position 1701 to the amino acid residue at position 1718 in SEQ ID NO:1.

7. The peptide of claim 5 which consists of the sequence having at least 60% of identity with the sequence which ranges from the amino acid residue position 1703 to the amino acid residue at position 1715 in SEQ ID NO:1.

8. The peptide of claim 1 which comprises the amino acid residues W1705, S1706, R1707, I1709, R1711, L1712, L1713, E1714.

9. The peptide of claim 1 which is fused to a carrier peptide.

10. The peptide of claim 1, selected from peptides of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO 24, SEQ ID NO 25, SEQ ID NO 26, SEQ ID NO 27, SEQ ID NO 28 or variants thereof.

11. A nucleic acid molecule encoding for the peptide of claim 1.

12. A vector which comprises the nucleic acid molecule of claim 11.

13. A host cell transformed with the nucleic acid molecule of claim 11.

14. An antibody or aptamer which specifically binds to the peptide of claim 1.

15-19. (canceled)

20. The peptide of claim 9, wherein the carrier peptide comprises a carrier peptide of sequence VKKKKIKAEIKI (SEQ ID NO: 29) or a carrier peptide of sequence THRPPMWSPVWP (SEQ ID NO: 30).

21. A method of treating a neurodegenerative disorder in a subject comprising administering to the subject an agent selected from the group consisting of the peptide of claim 1, a nucleic acid molecule encoding for the peptide, an aptamer which specifically binds to the peptide, or an antibody which specifically binds to the peptide.

22. The method according to claim 21, wherein the neurodegenerative disorder is an α-synucleinopathy, preferably selected from Parkinson's disease (PD), dementia with Lewy bodies (DLB), and multiple system atrophy (MSA).

23. The method according to claim 21, wherein the agent comprises the peptide of claim 1.


SEQUENCE LISTING (33 sequences)

SEQ ID NO: 1 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSASTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 2 - protein, 10 aa, Homo sapiens
INRLLEISPY

SEQ ID NO: 3 - protein, 11 aa, Homo sapiens
LINRLLEISPY

SEQ ID NO: 4 - protein, 13 aa, Homo sapiens
SRLINRLLEISPY

SEQ ID NO: 5 - protein, 9 aa, Homo sapiens
PMGFWSRLI

SEQ ID NO: 6 - protein, 16 aa, Homo sapiens
GFWSRLINRLLEISPY

SEQ ID NO: 7 - protein, 18 aa, Homo sapiens
PMGFWSRLINRLLEISPY

SEQ ID NO: 8 - protein, 13 aa, Homo sapiens
GFWSRLINRLLEI

SEQ ID NO: 9 - protein, 15 aa, Homo sapiens
PMGFWSRLINRLLEI

SEQ ID NO: 10 - protein, 21 aa, Artificial Sequence
  Synthetic construct seq_29 fused to seq_2
VKKKKIKAEIKINRLLEISPY

SEQ ID NO: 11 - protein, 23 aa, Artificial Sequence
  Synthetic construct seq_29 fused to seq_3
VKKKKIKAEIKILINRLLEISPY

SEQ ID NO: 12 - protein, 25 aa, Artificial Sequence
  Synthetic construct Seq_29 fused to seq_4
VKKKKIKAEIKISRLINRLLEISPY

SEQ ID NO: 13 - protein, 21 aa, Artificial Sequence
  Synthetic construct Seq_29 fused to seq_5
VKKKKIKAEIKIPMGFWSRLI

SEQ ID NO: 14 - protein, 30 aa, Artificial Sequence
  Synthetic construct Seq_29 fused to seq_7
VKKKKIKAEIKIPMGFWSRLINRLLEISPY

SEQ ID NO: 15 - protein, 25 aa, Artificial Sequence
  Synthetic construct Seq_29 fused to seq_8
VKKKKIKAEIKIGFWSRLINRLLEI

SEQ ID NO: 16 - protein, 28 aa, Artificial Sequence
  Synthetic construct Seq_29 fused to Seq_6
VKKKKIKAEIKIGFWSRLINRLLEISPY

SEQ ID NO: 17 - protein, 27 aa, Artificial Sequence
  Synthetic construct Seq_29 fused to Seq_9
VKKKKIKAEIKIPMGFWSRLINRLLEI

SEQ ID NO: 18 - protein, 22 aa, Artificial Sequence
  Synthetic construct Seq_30 fused to Seq_2
THRPPMWSPVWPINRLLEISPY

SEQ ID NO: 19 - protein, 23 aa, Artificial Sequence
  Synthetic construct Seq_30 fused to Seq_3
THRPPMWSPVWPLINRLLEISPY

SEQ ID NO: 20 - protein, 25 aa, Artificial Sequence
  Synthetic construct Seq_30 fused to Seq_4
THRPPMWSPVWPSRLINRLLEISPY

SEQ ID NO: 21 - protein, 21 aa, Artificial Sequence
  Synthetic construct Seq_30 fused to Seq_5
THRPPMWSPVWPPMGFWSRLI

SEQ ID NO: 22 - protein, 28 aa, Artificial Sequence
  Synthetic construct Seq_30 fused to Seq_6
THRPPMWSPVWPGFWSRLINRLLEISPY

SEQ ID NO: 23 - protein, 30 aa, Artificial Sequence
  Synthetic construct Seq_30 fused to Seq_7
THRPPMWSPVWPPMGFWSRLINRLLEISPY

SEQ ID NO: 24 - protein, 25 aa, Artificial Sequence
  Synthetic construct Seq_30 fused to Seq_8
THRPPMWSPVWPGFWSRLINRLLEI

SEQ ID NO: 25 - protein, 20 aa, Artificial Sequence
  Synthetic construct Seq_30 fused to Seq_5
THRPPMWSPVWPMGFWSRLI

SEQ ID NO: 26 - protein, 29 aa, Artificial Sequence
  Synthetic construct Seq_30 fused to Seq_7
THRPPMWSPVWPMGFWSRLINRLLEISPY

SEQ ID NO: 27 - protein, 26 aa, Artificial Sequence
  Synthetic construct Seq_30 fused to Seq_9
THRPPMWSPVWPMGFWSRLINRLLEI

SEQ ID NO: 28 - protein, 27 aa, Artificial Sequence
  Synthetic construct Seq 30 fused to Seq_9
THRPPMWSPVWPPMGFWSRLINRLLEI

SEQ ID NO: 29 - protein, 12 aa, Artificial Sequence
  Synthetic construct vectorization (or carrier) peptide disclosed in WO/2016/156536
VKKKKIKAEIKI

SEQ ID NO: 30 - protein, 12 aa, Artificial Sequence
  Synthetic construct peptide BBB shuttle THR from table 1 of Oller-Salvia et al (2016)
THRPPMWSPVWP

SEQ ID NO: 31 - protein, 12 aa, Artificial Sequence
  Synthetic construct vectorization (or carrier) peptide disclosed in WO/2016/156536
VKKKKIKREIKI

SEQ ID NO: 32 - protein, 12 aa, Artificial Sequence
  Synthetic construct vectorization (or carrier) peptide disclosed in WO/2016/156536
VKKKKIKKEIKI

SEQ ID NO: 33 - protein, 12 aa, Artificial Sequence
  Synthetic construct vectorization (or carrier) peptide disclosed in WO/2016/156536
VKKKKIKNEIKI